(19) United States
(12) Patent Application Publication
(10) Pub. No.: US 2016/0151193 A1
(43) Pub. Date: Jun. 2, 2016

(54) PORTABLE CERVICAL TRACTION DEVICE WITH SUSPENSION HEAD HALTER

(71) Applicant: Hollywog, LLC, Chattanooga, TN (US)

(72) Inventors: Charles M. Thomas, Hixson, TN (US); D. Keith Hagy, Hixson, TN (US); Thu-Ha Thi Duncan, Cleveland, TN (US); John S. Marshall, Harrison, TN (US); Guy T. Hill, Rock Springs, GA (US)

(73) Assignee: Hollywog, LLC, Chattanooga, TN (US)

(21) Appl. No.: 15/016,721

(22) Filed: Feb. 5, 2016

Related U.S. Application Data
(63) Continuation-in-part of application No. 14/801,453, filed on Jul. 16, 2015.
(60) Provisional application No. 62/025,860, filed on Jul. 17, 2014.

(51) Int. Cl. A61F 5/048; A61F 5/055; A61F 5/37

(52) U.S. Cl. CPC A61F 5/048; A61F 5/3707; A61F 5/055

(57) ABSTRACT

A cervical traction device includes a base assembly having a sled frame and a sled assembly that is adapted to move along the sled frame. The cervical traction device includes a left halter support that is attached to the sled assembly and a right halter support that is attached to the sled assembly. A head halter is adapted to be removably attached to the left halter support and the right halter support. The head halter includes a suspension strap for supporting the patient's neck. A linear actuator is attached between the base assembly and the sled assembly and adapted to move the sled assembly along the sled frame.

### CROSS-REFERENCE TO RELATED APPLICATIONS

This application is a continuation-in-part of U.S. patent application Ser. No. 14/801,453, filed Jul. 16, 2015, which application claims the benefit of U.S. Provisional Patent Application No. 62/025,860 that was filed on Jul. 17, 2014.

### FIELD OF THE INVENTION

This invention relates generally to cervical traction devices. More specifically, the invention relates to a portable cervical traction device that may be placed on any suitable surface for use in applying traction forces to the cervical musculature and spine, and to a head halter for a portable cervical traction device that includes an occipital suspension strap.

### BACKGROUND AND DESCRIPTION OF THE PRIOR ART

Traction devices are commonly used to relieve pressure on inflamed or pinched nerves. Cervical traction devices generally operate by applying a traction force to the head and/or neck of a patient, and lumbar traction devices generally operate by applying a traction force to the lumbar spine. Such devices can relieve pain in the neck and lower back by stretching the cervical and/or lumbar musculature and spine. Generally, traction devices operate by applying a series of traction or pulling forces to the head and/or neck or to the hips while the rest of the body is held down by straps or by friction forces on a table or other surface.

Portable traction devices are known for allowing patients to perform traction therapy on themselves in the privacy of their homes, or for allowing therapists to perform traction therapy in a clinical setting by placing the portable device on any of various inexpensive support tables or even on the floor. Common portable cervical traction devices apply traction forces by means of pneumatic cylinders that are controlled by a hand pump, spring-loaded linear actuators or motorized linear actuators. Such traction devices do not typically provide support for the patient's shoulders in such a way as to help to position the patient properly with respect to the traction apparatus. In addition, conventional cervical traction devices do not include a self-adjusting halter assembly with a suspension strap for properly positioning the head and neck of a patient with respect to the patient's shoulders to obtain a maximum therapeutic benefit.

It would be desirable if a cervical traction device could be provided that includes multiple interchangeable self-storing head support cushions for use in positioning the patient's head and neck in any of several angular positions with respect to the patient's shoulders. It would also be desirable if the cervical traction device included a shoulder support assembly that assists in properly positioning the patient to receive traction therapy. It would also be desirable if the cervical traction device included a halter assembly that includes an occipital suspension strap, which assembly cooperates with the shoulder support assembly and one of the head support cushions for properly positioning the cervical spine of a patient to obtain a maximum therapeutic benefit.

### Advantages of a Preferred Embodiment of the Invention

Among the advantages of a preferred embodiment of the invention is that it provides a cervical traction device having multiple interchangeable self-storing head support cushions for use in positioning the patient's head and neck in any of several angular positions with respect to the patient's shoulders. Another advantage of a preferred embodiment of the invention is that it includes a support component for the patient's shoulders that assists in properly positioning the patient to receive traction therapy. Still another advantage of a preferred embodiment of the invention is that it includes a halter assembly which includes a suspension strap, which halter assembly cooperates with the shoulder support component and the selected head support cushion for properly positioning the cervical spine of a patient to obtain a maximum therapeutic benefit.

Additional objects and advantages of this invention will become apparent from an examination of the drawings and the ensuing description.

### NOTES ON CONSTRUCTION

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic. All methods described herein can be performed in any suitable order unless otherwise specified herein or clearly indicated by context.

The use of any and all examples or exemplary language (e.g., “such as” and “preferably”) herein is intended merely to better illuminate the invention and the preferred embodiments thereof, and not to place a limitation on the scope of the invention. Nothing in the specification should be construed as indicating any element as essential to the practice of the invention unless so stated with specificity. Several terms are specifically defined herein. These terms are to be given their broadest possible construction consistent with such definitions, as follows:

The terms “above”, “upper” and similar terms, when used with respect to a traction device or a component of a traction device, refer to a relative location or direction away from the surface on which the device is placed during operation.

The terms “below”, “lower” and similar terms, when used with respect to a traction device or a component of a traction device, refer to a relative location or direction towards the surface on which the device is placed during operation.

The term “rear” and similar terms, when used with respect to a traction device or a component of a traction device, refer to a relative location or direction towards the end of the device on which the shoulder support cushion is located.

The terms “front”, “forward” and similar terms, when used with respect to a traction device or a component of a traction device, refer to a relative location or direction away from the end of the device on which the shoulder support cushion is located.

The term “linear actuator” refers to an electric, hydraulic, electro-hydraulic or mechanical device that generates force which is directed in a straight line.

### SUMMARY OF THE INVENTION

The invention comprises a cervical traction device for use in applying cervical traction to a patient. This cervical traction device includes a base assembly and a sled assembly. The base assembly has a sled frame, and the sled assembly is adapted to move along the sled frame. A linear actuator is attached between the base assembly and the sled assembly, and adapted to move the sled assembly along the sled frame. The invention includes a halter assembly having a head halter that is adapted to be removably attached to the head of the patient and which comprises a suspension strap for supporting the patient's neck.

The preferred halter assembly includes left and right halter supports which are attached on opposite sides of the sled assembly, and the head halter is adapted to be removably attached to the halter supports. The suspension strap of the preferred head halter is adapted to be positioned at or slightly beneath the occiput for supporting the neck of the patient. The preferred embodiment of the invention allows for the application of a traction force to a patient by movement of the sled assembly with respect to the base assembly that will provide a release of the cervical spine. The preferred halter assembly is sized and configured so as to be self-adjusting to accommodate patients of various sizes and weights.

In a preferred embodiment of the invention, the sled assembly has a recess that is adapted to removably receive each of a plurality of head support cushions. Each of these head support cushions has a configuration that is different from the configurations of the other head support cushions. The preferred cervical traction device also includes a shoulder support cushion. Preferably, this shoulder support cushion is removably attached to the base assembly and includes a storage slot that is adapted to securely hold one or more of the head support cushions.

In the preferred embodiment of the invention, the shoulder support cushion, head support cushions and halter assembly cooperate to position the head and neck of the patient with respect to the patient's shoulders to facilitate the application of a therapeutic traction force.

In order to facilitate an understanding of the invention, the preferred embodiments of the invention is illustrated in the drawings, and a detailed description thereof follows. It is not intended, however, that the invention be limited to the particular embodiments described or to use in connection with the apparatus illustrated herein. Various modifications and alternative embodiments such as would ordinarily occur to one skilled in the art to which the invention relates are also contemplated and included within the scope of the invention described and claimed herein.

### BRIEF DESCRIPTION OF THE DRAWINGS

The presently preferred embodiments of the invention are illustrated in the accompanying drawings, in which like reference numerals represent like parts throughout, and in which:

FIG. 1 is a perspective view of a preferred embodiment of the cervical traction device of the invention, with the head halter removed to more clearly reveal the features of the halter supports.

FIG. 2 is a top view of the embodiment of the cervical traction device shown in FIG. 1.

FIG. 3 is a side view of the embodiment of the cervical traction device shown in FIGS. 1 and 2.

FIG. 4 is an end view of the embodiment of the cervical traction device shown in FIGS. 1-3, taken along line 4-4 of FIG. 3.

FIG. 5 is an end view of the embodiment of the cervical traction device shown in FIGS. 1-4, taken along line 5-5 of FIG. 3.

FIG. 6 is an exploded view, in perspective, of the cervical traction device of the invention shown in FIGS. 1-5.

FIG. 7 is an exploded view, in perspective, of a portion of the base assembly of the cervical traction device shown in FIGS. 1-6.

FIG. 8 is an exploded view, in perspective, of the sled assembly of the cervical traction device shown in FIGS. 1-6.

FIG. 9A is a top view of a first head support cushion that is included within a preferred embodiment of the invention.

FIG. 9B is a side view of the head support cushion shown in FIG. 9A.

FIG. 9C is an end view of the head support cushion shown in FIGS. 9A and 9B.

FIG. 9D is a perspective view of the head support cushion shown in FIGS. 9A-9C.

FIG. 9E is a bottom view of the head support cushion shown in FIGS. 9A-9D.

FIG. 9F is a sectional view of the head support cushion shown in FIGS. 9A-9E, taken along line F-F of FIG. 9E.

FIG. 10A is a top view of a second head support cushion that is included within a preferred embodiment of the invention.

FIG. 10B is a side view of the head support cushion shown in FIG. 10A.

FIG. 10C is an end view of the head support cushion shown in FIGS. 10A and 10B.

FIG. 10D is a perspective view of the head support cushion shown in FIGS. 10A-10C.

FIG. 10E is a bottom view of the head support cushion shown in FIGS. 10A-10D.

FIG. 10F is a sectional view of the head support cushion shown in FIGS. 10A-10E, taken along line F-F of FIG. 10E.

FIG. 11A is a top view of a third head support cushion that is included within a preferred embodiment of the invention.

FIG. 11B is a side view of the head support cushion shown in FIG. 11A.

FIG. 11C is an end view of the head support cushion shown in FIGS. 11A and 11B.

FIG. 11D is a perspective view of the head support cushion shown in FIGS. 11A-11C.

FIG. 11E is a bottom view of the head support cushion shown in FIGS. 11A-11D.

FIG. 11F is a sectional view of the head support cushion shown in FIGS. 11A-11E, taken along line F-F of FIG. 11E.

FIG. 12A is a top view of the preferred shoulder support cushion that is a part of the invention.

FIG. 12B is a side view of the shoulder support cushion shown in FIG. 12A.

FIG. 12C is a front end view of the shoulder support cushion shown in FIGS. 12A and 12B.

FIG. 12D is a perspective view of the shoulder support cushion shown in FIGS. 12A-12C.

FIG. 12E is a bottom view of the shoulder support cushion shown in FIGS. 12A-12D.

FIG. 12F is a sectional view of the shoulder support cushion shown in FIGS. 12A-12E, taken along line F-F of FIG. 12A.

FIG. 12G is a sectional view of the shoulder support cushion shown in FIGS. 12A-12F, taken along line G-G of FIG. 12E.

FIG. 12H is a sectional view of the shoulder support cushion shown in FIGS. 12A-12G, taken along line H-H of FIG. 12E.

FIG. 13A illustrates a first embodiment of a head halter that is a part of the preferred embodiment of the invention.

FIG. 13B illustrates the head halter shown in FIG. 13A, showing how it is attached to a patient and to the cervical traction device shown in FIGS. 1-6.

FIG. 14A illustrates a second embodiment of a head halter that is a part of the preferred embodiment of the invention.

FIG. 14B illustrates the head halter shown in FIG. 14A, showing how it is attached to a patient and to the cervical traction device shown in FIGS. 1-6.

FIG. 15A illustrates a third embodiment of a head halter that is a part of the preferred embodiment of the invention.

FIG. 15B illustrates the components of the head halter shown in FIG. 15A, showing how it is attached to the cervical traction device shown in FIGS. 1-6.

FIG. 15C illustrates the head halter shown in FIGS. 15A and 15B, showing how it is attached to a patient and to the cervical traction device shown in FIGS. 1-6.

### DESCRIPTION OF THE PREFERRED EMBODIMENT OF THE INVENTION

A preferred embodiment 20 of a cervical traction device is illustrated in the drawings. As shown therein, cervical traction device 20 includes base assembly 22, sled assembly 24, left and right halter supports 26 and 28 respectively, and shoulder support cushion 30.

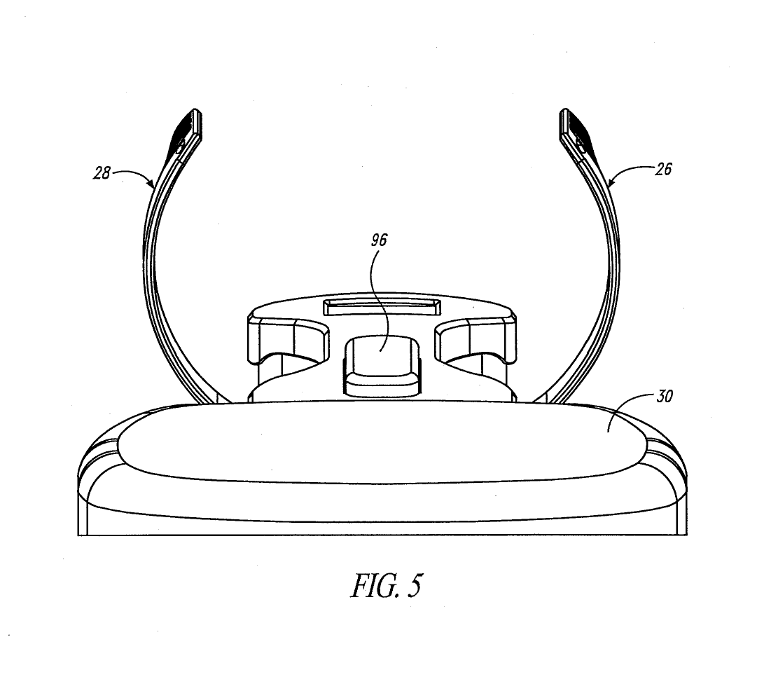

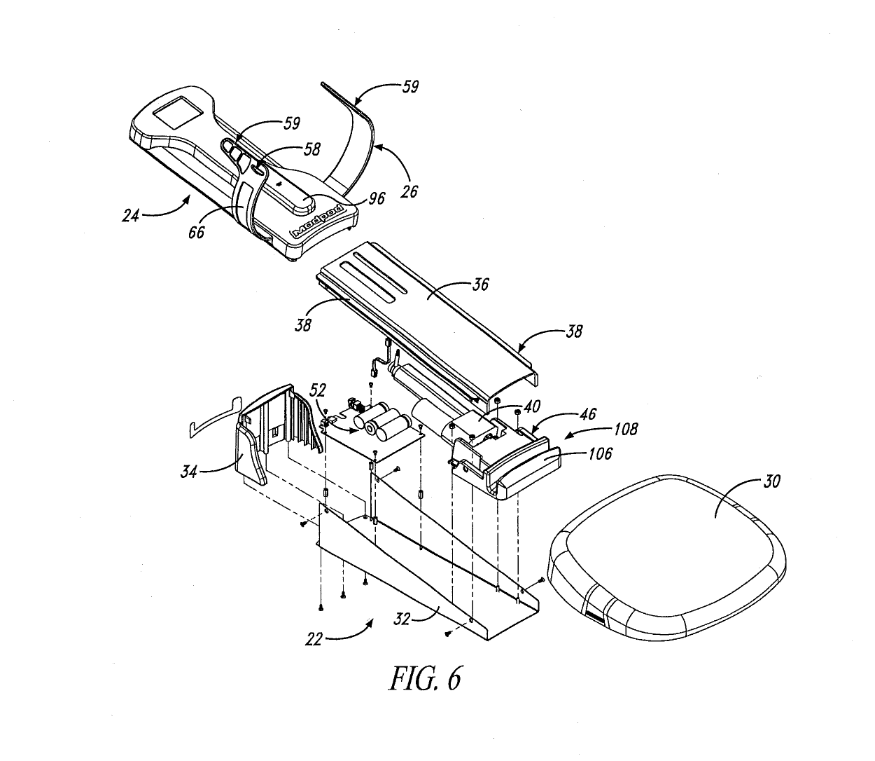

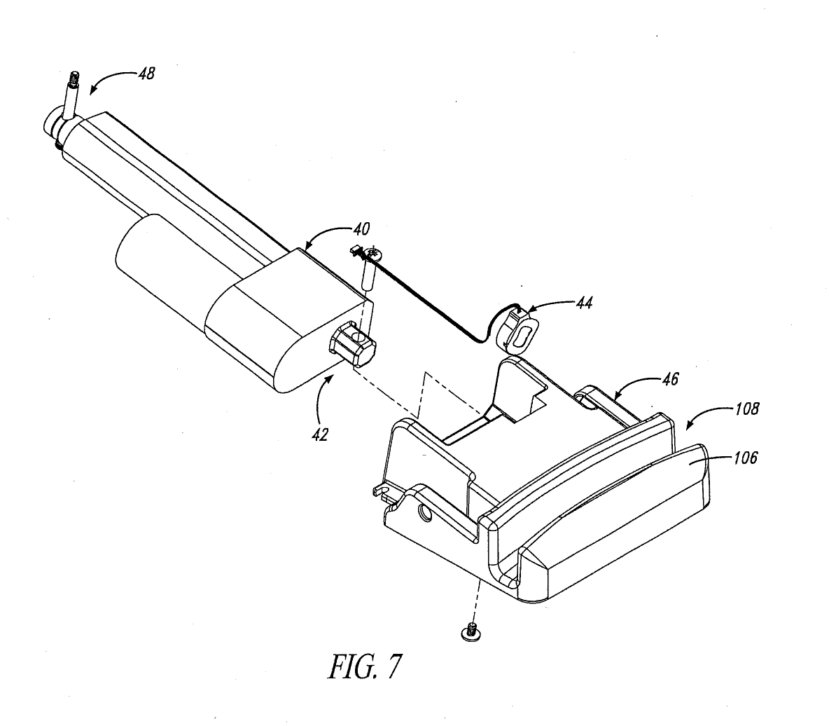

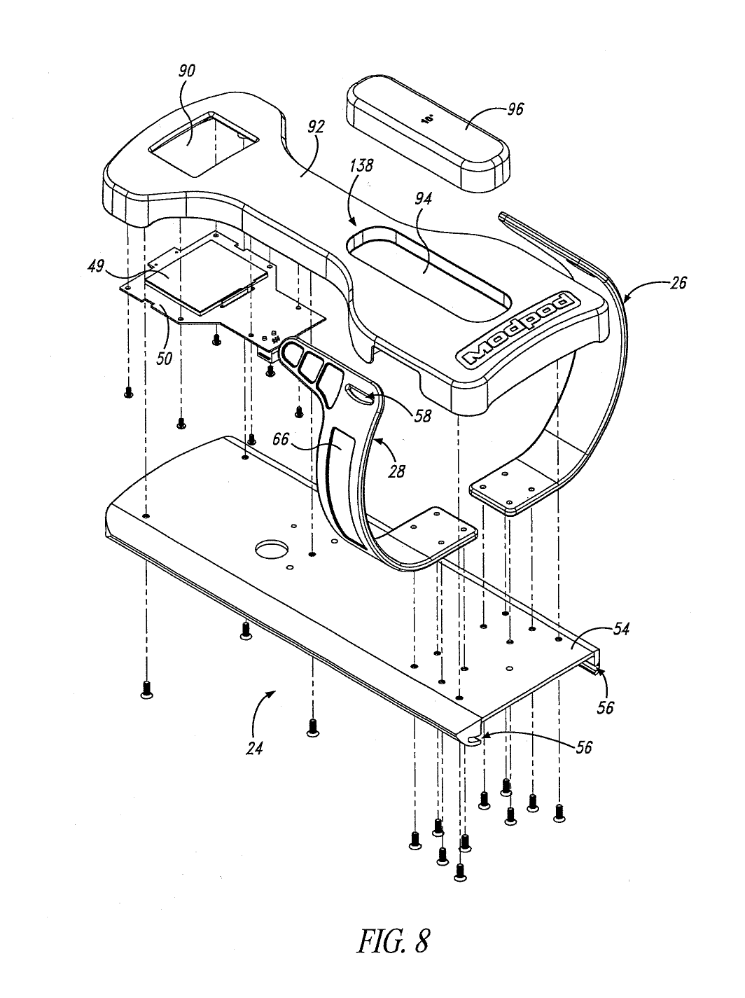

As shown in FIG. 6, base assembly 22 includes base frame 32, front closure 34, and sled frame 36. Two low-friction sled runners 38 are attached to the sled frame to facilitate movement of sled assembly 24 on the sled frame. Preferably, sled runners 38 are comprised of a blend of polycarbonate and glass fibers. Enclosed within base assembly 22 is linear actuator 40 (best shown in FIG. 7). Rear end 42 of linear actuator 40 is attached to load cell assembly 44 and to rear support 46 of the base assembly. Front end 48 of linear actuator 40 is attached to sled assembly 24. The load cell assembly is operatively attached to the linear actuator and to controller and interactive display assembly 49 located on controller base 50 (shown in FIG. 8) in sled assembly 24. Actuator 40 is powered by rechargeable battery pack 52 located in base frame 32.

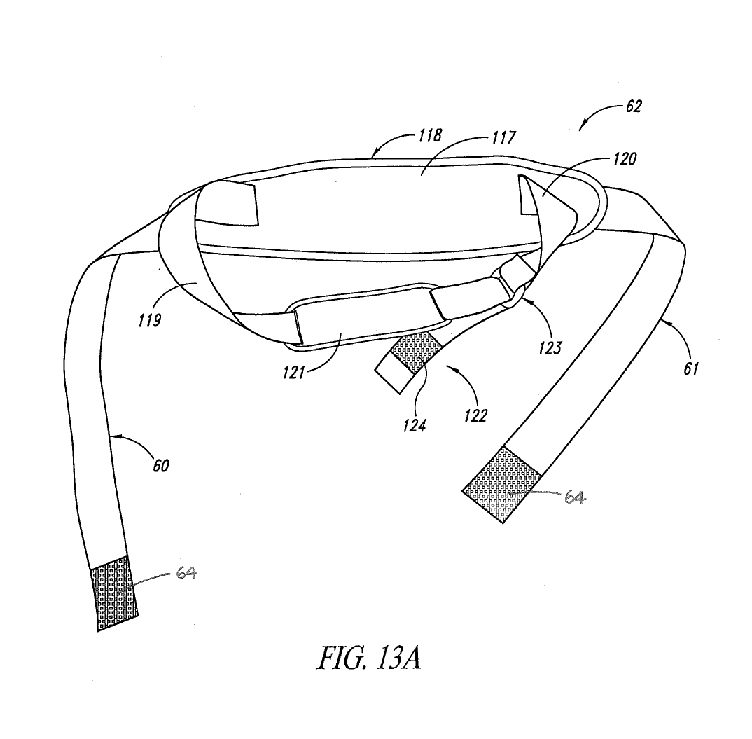

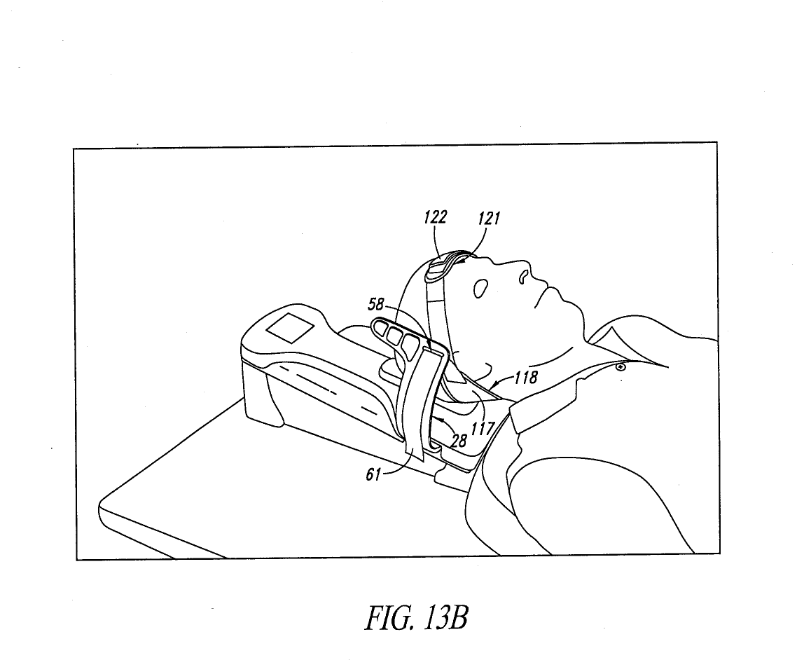

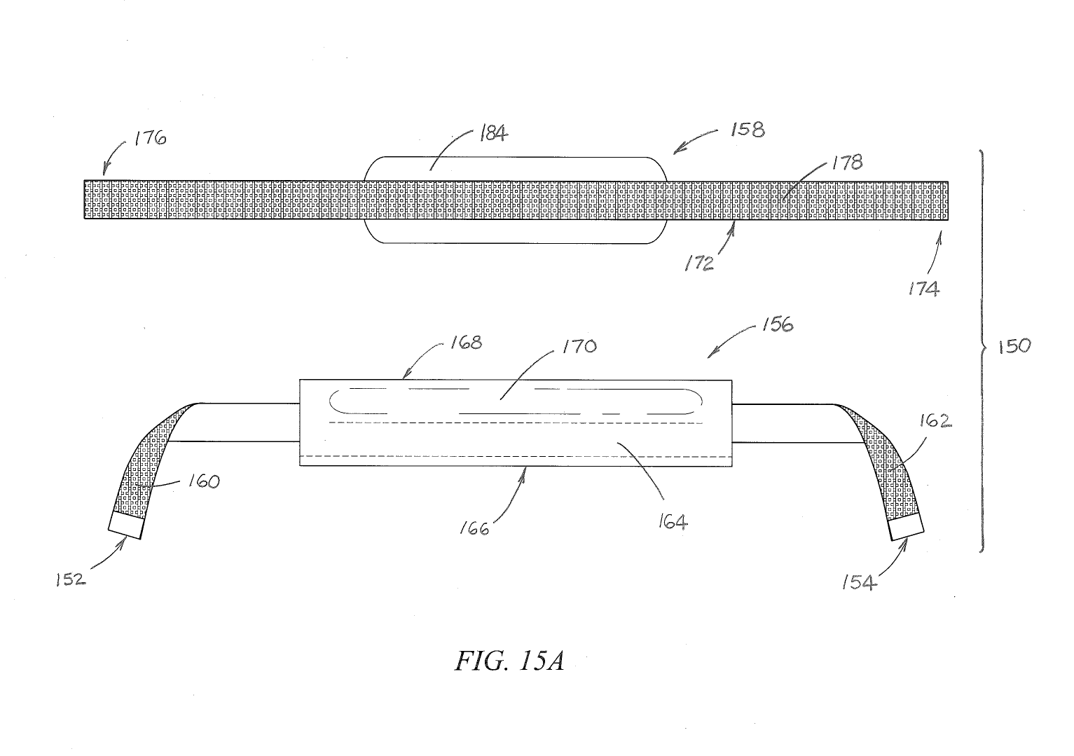

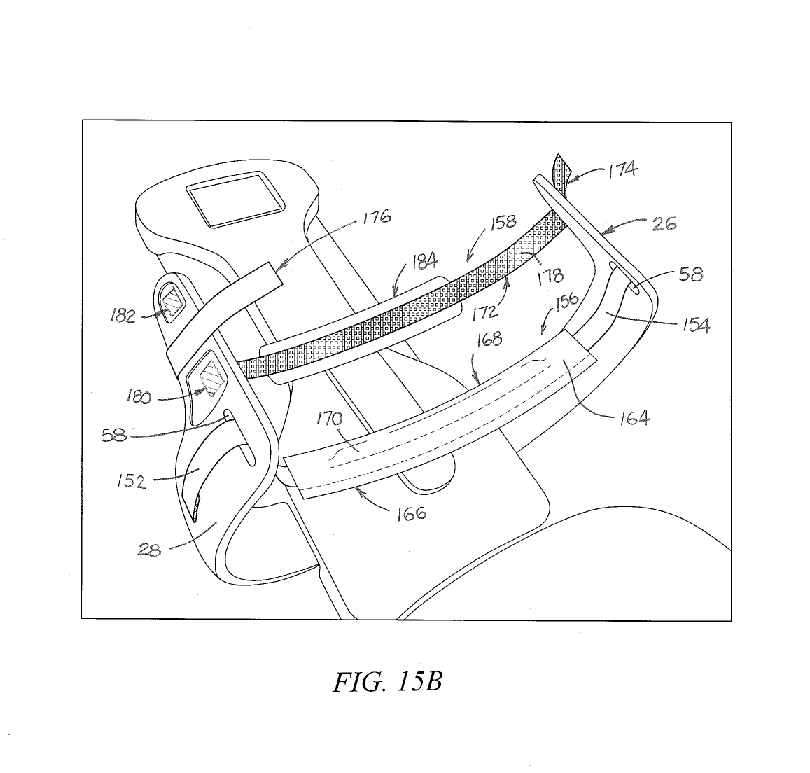

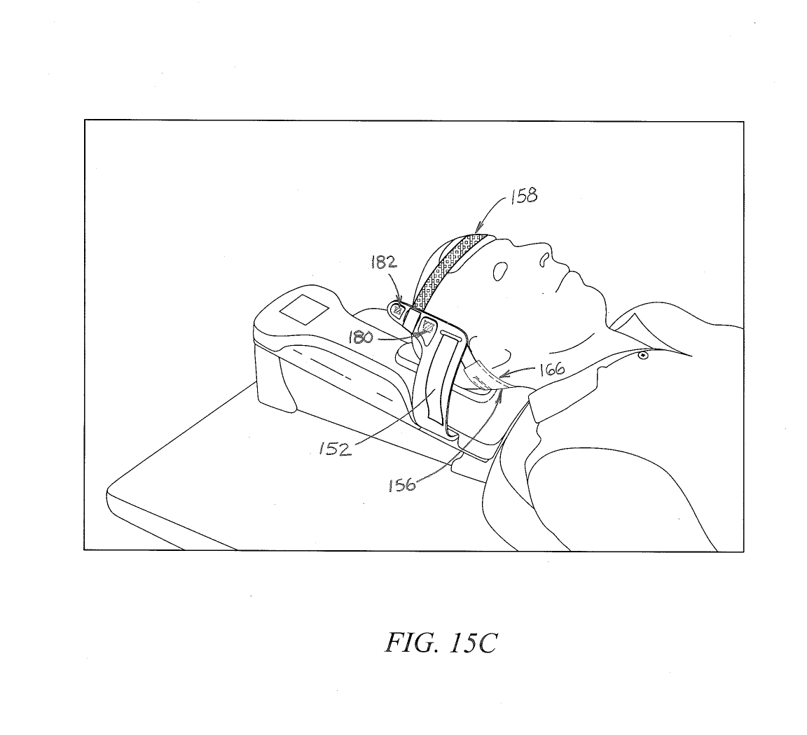

As shown in FIG. 8, sled assembly 24 includes sled base 54, which is provided with a pair of slots 56 for engaging sled runners 38 on sled frame 36. Attached to the sled base is left halter support 26 and right halter support 28. Each of the halter supports has a halter support slot 58 and a front projection 59. Each of halter support slots 58 is adapted to receive an end strap 60 or 61 of head halter 62 (shown in FIGS. 13A and 13B), or an end strap 152 or 154 of suspension strap 156 of head halter 150 (shown in FIGS. 15A, 15B and 15C).

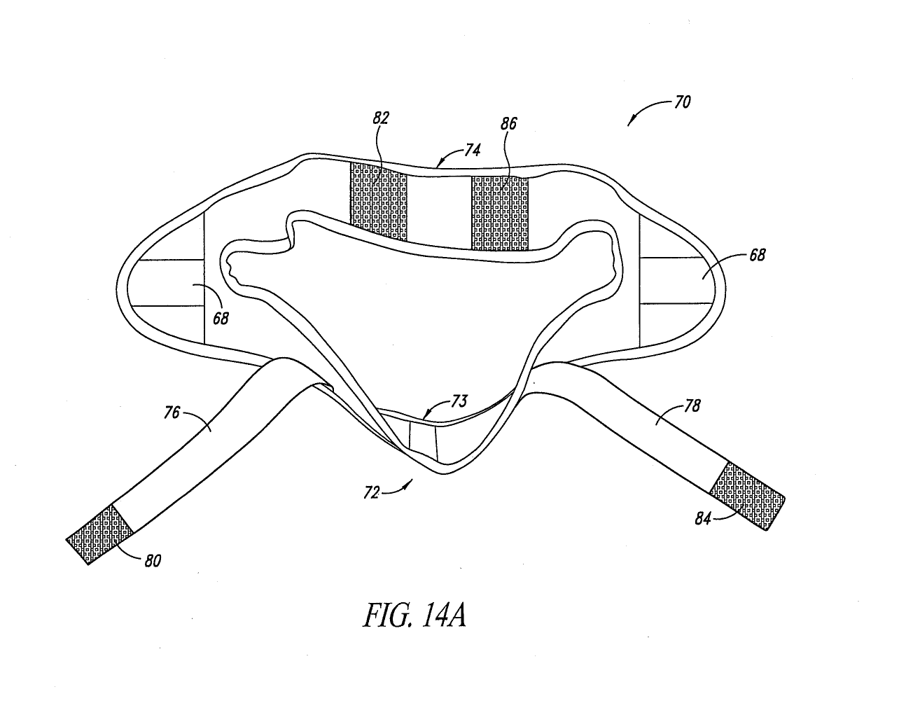

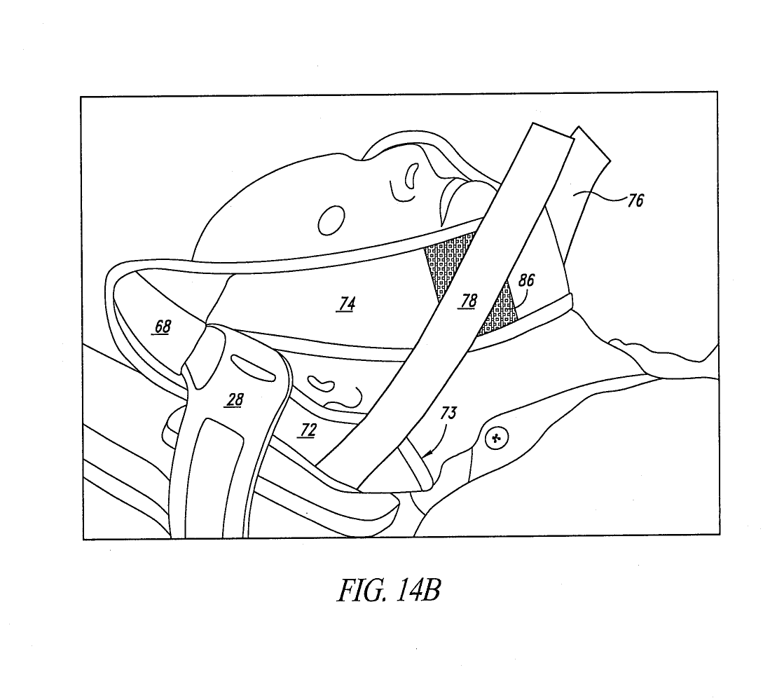

Each of end straps 60 and 61 of head halter 62 is preferably provided with a portion 64 of a hook and loop fastener, and this portion of the hook and loop fastener engages a mating portion 66 of a hook and loop fastener located on left halter support 26 and right halter support 28 to hold head halter 62 in place with respect to the halter supports. Similarly, each of end straps 152 and 154 of suspension strap 156 of head halter 150 is preferably provided with a portion 158 of a hook and loop fastener, and this portion of the hook and loop fastener engages mating portion 66 of a hook and loop fastener located on left halter support 26 and right halter support 28 to hold suspension strap 156 of head halter 150 in place with respect to the halter supports. Each of front projections 59 on left halter support 26 and right halter support 28 is adapted to receive cupped end 68 of head halter 70 (shown in FIGS. 14A and 14B).

Controller and interactive display assembly 49 allows a user of cervical traction device 20 to select from and modify any of various programmed clinical protocols or to program additional clinical protocols for the application of traction forces, and to operate device 20 to apply the selected traction forces to a patient. The interactive display portion of assembly 49 is visible through window 90 of sled top 92 when the sled top is attached to sled base 54. In this embodiment of the invention, controller and interactive display assembly 49 continuously measures the distance traveled by the sled and cooperates with load cell assembly 44 to measure the load or traction force applied by the linear actuator to determine the proper application of traction forces for maximum therapeutic effect, as set by means of the interactive display portion of assembly 49. This allows cervical traction device 20 to continuously apply the desired amount of traction force, even if the patient moves his or her head during treatment.

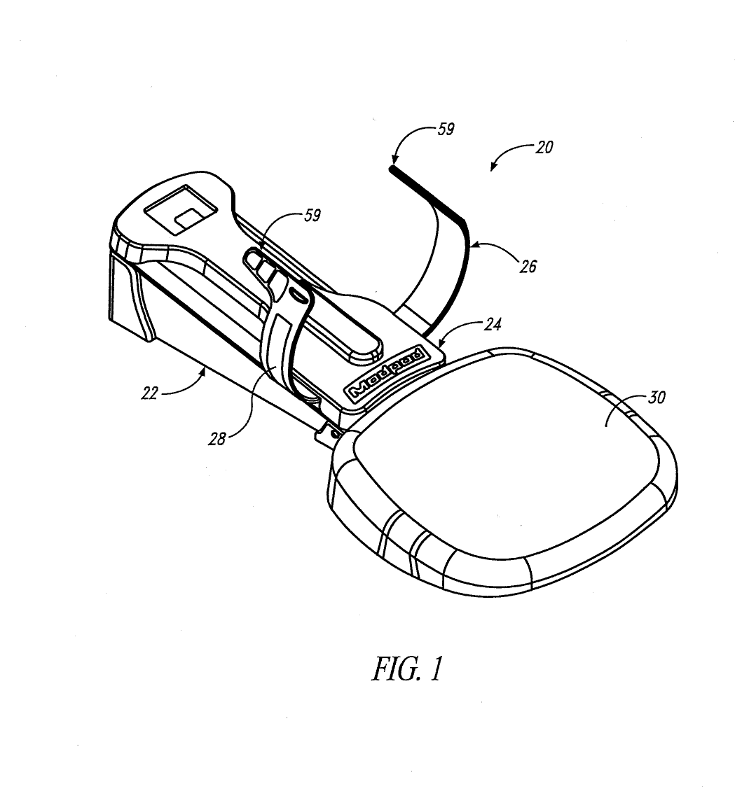

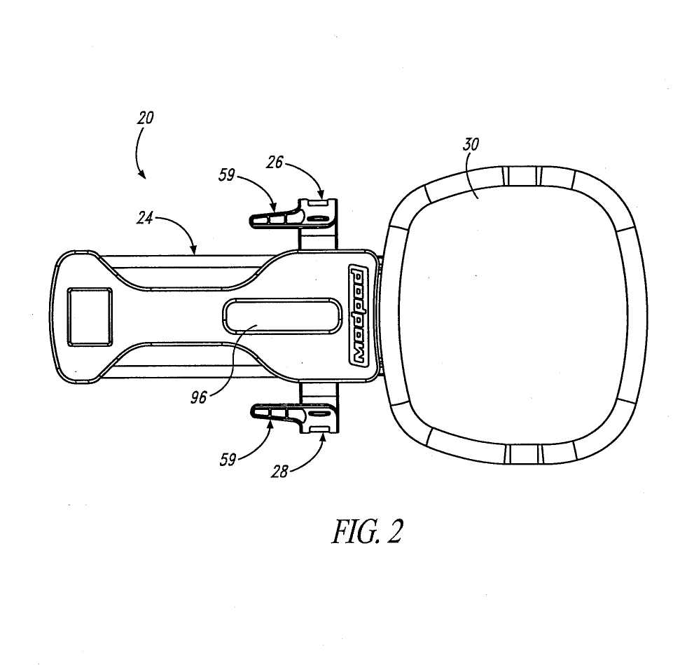

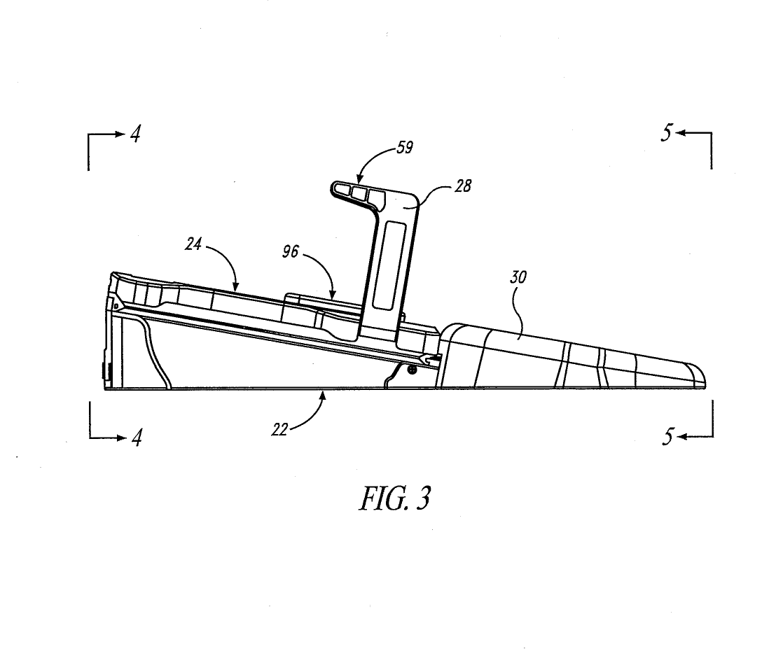

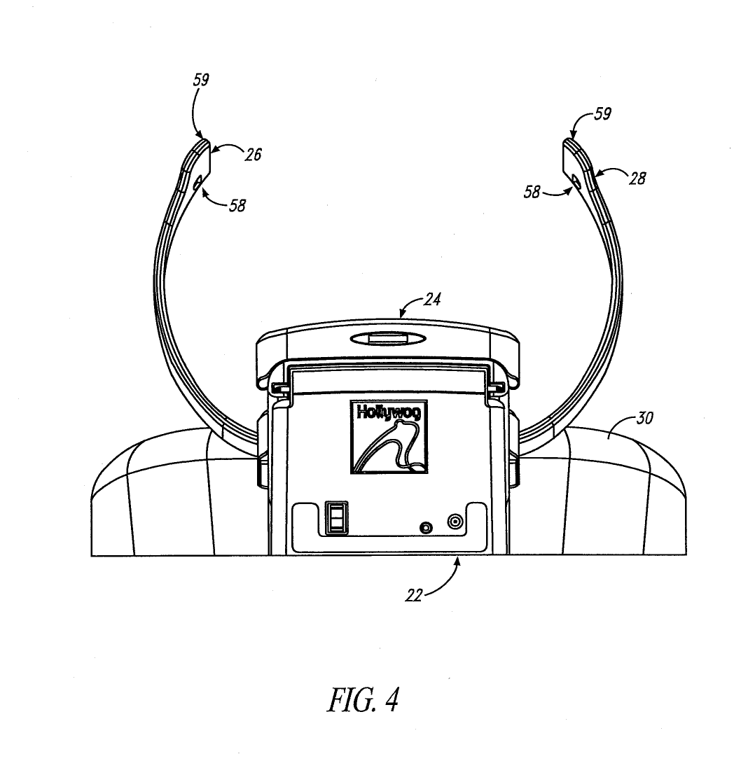

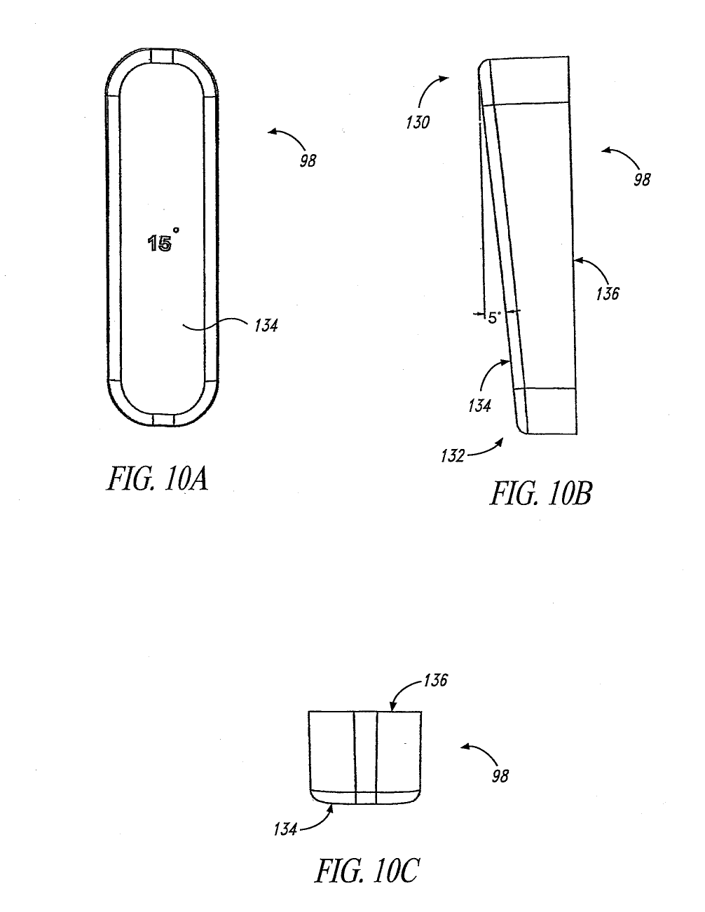

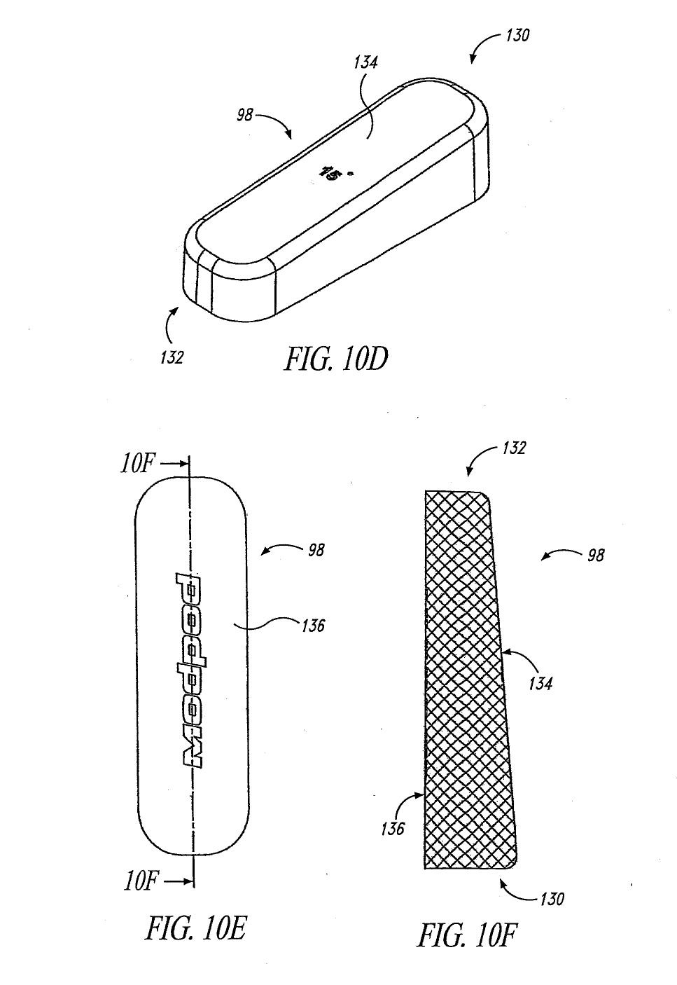

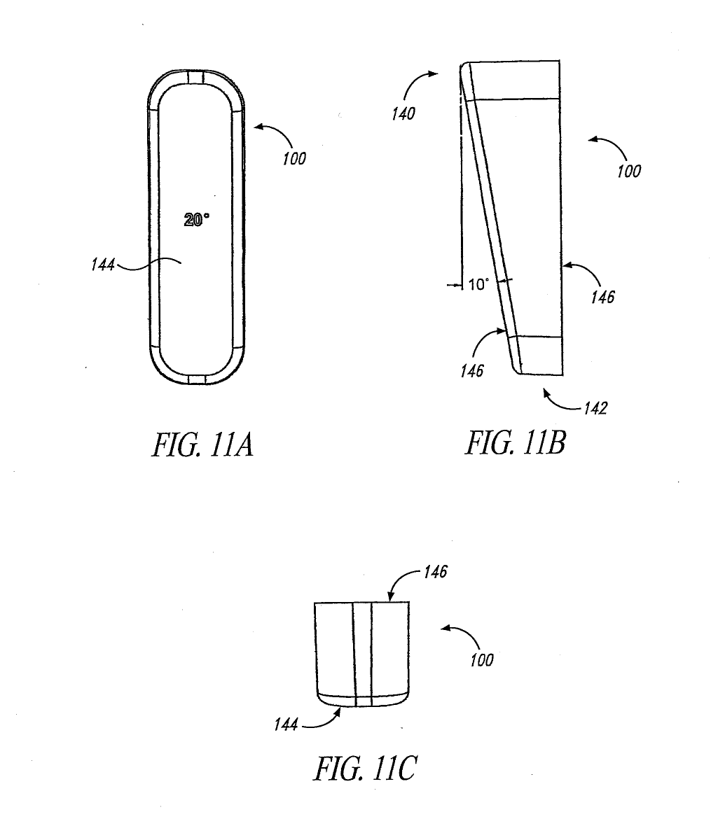

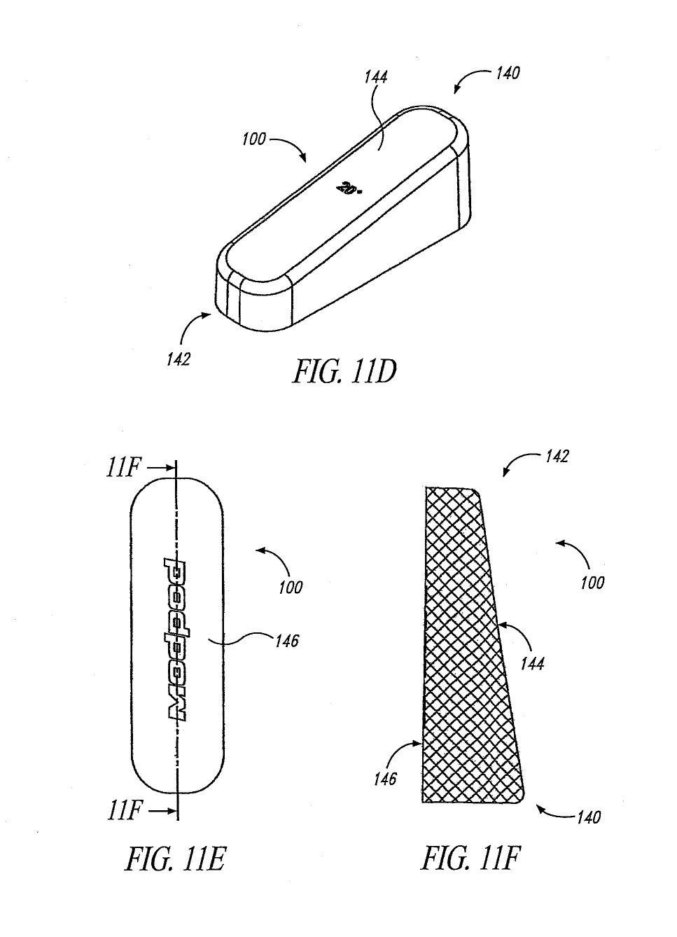

Also provided in sled top 92 is cut-out 94 which forms, in cooperation with sled base 54, a recess in the sled assembly that is adapted to removably receive any of several head support cushions, such as head support cushion 96 shown in FIGS. 1-3, 5, 6, 8 and 9A-9E, head support cushion 98 shown in FIGS. 10A-10F and head support cushion 100 shown in FIGS. 11A-11F.

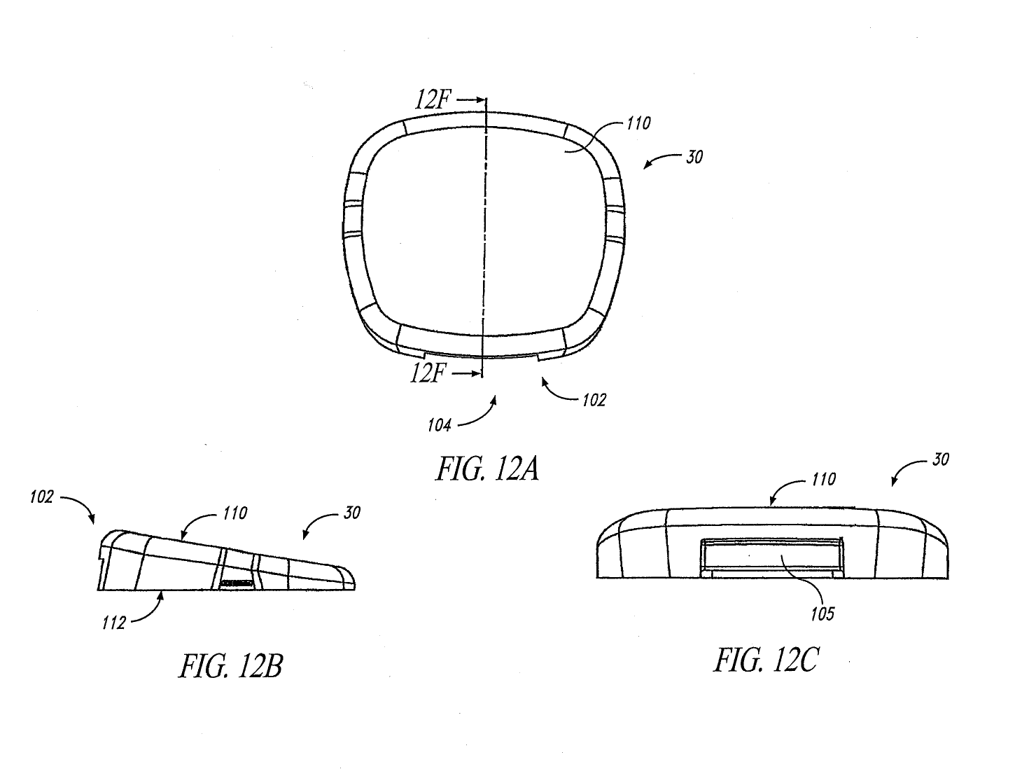

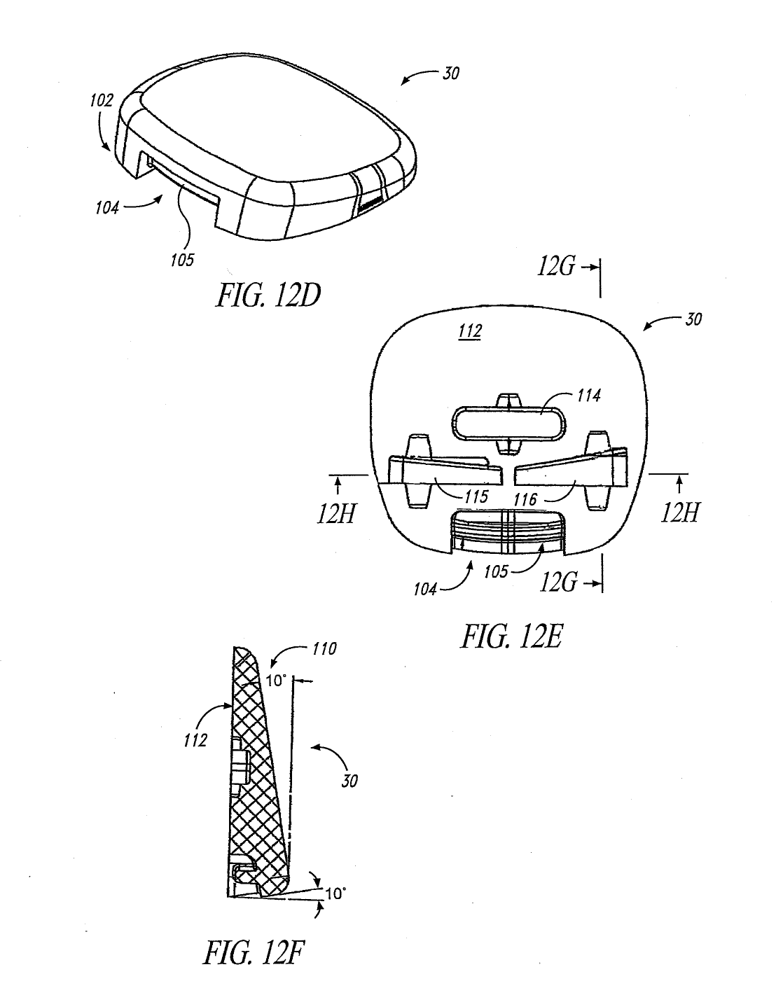

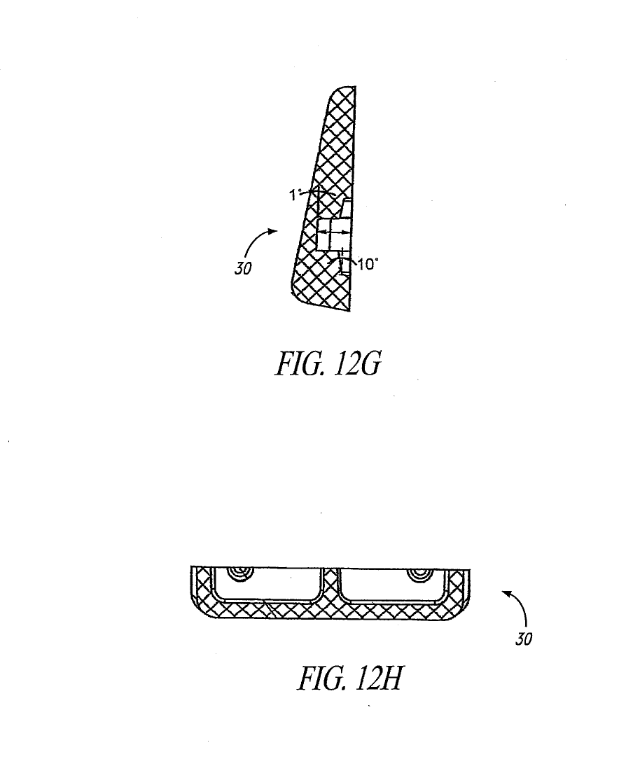

The invention also includes removable shoulder support cushion 30 having a front end 102 with an opening 104 and a base engagement projection 105 recessed in the opening, as shown in FIGS. 12A-12E. FIGS. 6 and 7 illustrate nose portion 106 and slot 108 of rear support 46 of base assembly 22. By comparing FIGS. 6, 7 and 12A-12E, it can be seen that base engagement projection 105 is sized and configured to be received in slot 108 in front of nose portion 106 of rear support 46 to securely hold the shoulder support cushion in engagement with the base assembly with the shoulder support cushion resting on the surface on which device 20 is placed. The upper surface 110 of shoulder support cushion is inclined upwardly towards front end 102, preferably by 10° with respect to lower surface 112, as best shown in FIG. 12F. Lower surface 112 also includes storage slot 114 that is adapted to securely hold head support cushion 96, storage slot 115 that is adapted to securely hold head support cushion 98, and storage slot 116 that is adapted to securely hold head support cushion 100, when the head support cushions are not placed in the recess defined in party by cut-out 94.

The invention includes a self-adjusting halter assembly for properly positioning the head and neck of a patient with respect to the patient's shoulders to obtain a maximum therapeutic benefit. The halter assembly is also sized and configured so as to be self-adjusting to accommodate patients of various sizes and weights. The halter assembly comprises left halter support 26, right halter support 28 and a head halter. Three different head halters are described herein, and all are characterized by a suspension strap that is adapted to support the patient's neck and lower skull at or slightly beneath the occiput. The suspension strap of each head halter preferably suspends at least a portion of the patient's neck so that it does not contact the shoulder support or the head support cushion that is in use. Preferably, the suspension strap of each head halter suspends at least a portion of the patient's neck and lower skull so that these portions of the patient's anatomy do not contact any other portion or component of the halter assembly or the cervical traction device. Head halter 62 is particularly adapted for use by patients with temporomandibular joint (“TMJ”) disorders or other disorders that would cause discomfort if a head halter included a chin strap. Head halter 70 is adapted for use by patients who can easily tolerate a chin strap, and head halter 150 can be used by most patients.

Head halter 62 includes left end strap 60 and right end strap 61, neck suspension strap 117 having rear edge 118, and an adjustable forehead support strap comprising left side portion 119 and right side portion 120, both of which are attached to the neck suspension strap. Left side portion 119 also includes forehead pad 121 and free end 122. Right side portion 120 terminates in ring 123. Free end 122 of left side portion 119 is provided with a portion 124 of a hook and loop fastener that is adapted to engage and mate with a portion of a hook and loop fastener (not shown) located on the left side portion 119 adjacent forehead pad 121 after free end 122 is passed through ring 123. The proper placement of head halter 62 on the left and right halter supports is shown in FIG. 13B, with the end straps (only one of which, end strap 61, is shown in FIG. 13B) being passed through halter support slots 58 in the halter supports and secured to the halter supports as described above. Left halter support 26, right halter support 28 and head halter 62 are sized and configured so that when end straps 60 and 61 of head halter 62 are placed through halter support slots 58 in the halter supports (as shown in FIG. 13B) and secured to the halter supports, and the patient is positioned so that rear edge 118 of neck suspension strap 117 is at or slightly beneath the occiput and the free end 122 of left side portion 119 of the adjustable forehead support strap is passed through ring 123 and secured around the patient's forehead, at least a portion of the patient's neck and lower skull are suspended so that these portions of the patient's anatomy do not contact any other portion or component of the halter assembly or the cervical traction device. In this circumstance, the application of a traction force to the patient by movement of sled assembly 24 on the sled frame will provide a C1-C7 release of the cervical spine.

Head halter 70 also includes suspension strap 72 having rear edge 73, chin strap 74, and a pair of fastener straps 76 and 78. Fastener strap 76 is provided with a portion 80 of a hook and loop fastener, and this portion of the hook and loop fastener engages a mating portion 82 of a hook and loop fastener located on chin strap 74. Similarly, fastener strap 78 is provided with a portion 84 of a hook and loop fastener, and this portion of the hook and loop fastener engages a mating portion 86 of a hook and loop fastener located on chin strap 74. After the cupped ends 68 of head halter 70 are slipped over front projections 59 on the halter supports, the patient is positioned so that rear edge 73 of suspension strap 72 is at or slightly beneath the occiput and chin strap 74 is placed under the patient's jaw. Then, the fastener straps are attached to the chin strap to snugly hold the patient's head. Head halter 70 serves to suspend at least a portion of the patient's neck and lower skull so that these portions of the patient's anatomy do not contact any other portion or component of the halter assembly or the cervical traction device. Thus, the head halter places the patient's head and neck in a suitable position for the application of traction therapy.

Head halter 150 comprises suspension strap 156 and forehead strap 158, as shown in FIGS. 15A, 15B and 15C. Suspension strap 156 includes right end strap portion 152 which is placed through right halter support slot 58 in right halter support 28 and is preferably provided with a portion 160 of a hook and loop fastener that is adapted to engage mating portion 66 of a hook and loop fastener located on right halter support 28 (see FIGS. 6 and 8). Similarly, strap 156 includes left end strap portion 154 which is placed through left halter support slot 58 in left halter support 26 and is preferably provided with a portion 162 of a hook and loop fastener that is adapted to a engage mating portion of a hook and loop fastener located on left halter support 26 (not shown but substantially similar to portion 66 of hook and loop fastener located on right halter support 28). This structure acts to hold suspension strap 156 of head halter 150 in place with respect to the halter supports. Attached to suspension strap 156 is neck pad 164 which is preferably made of an anti-slip fabric material, such as coated neoprene. Most preferably, neck pad 164 has a rear edge 166 and a front edge 168, and it includes a semi-rigid flexible material 170 (shown in outline in the drawings), such as closed cell neoprene material, which is sewn into the anti-slip material near front edge 168.

Head halter 150 also includes forehead strap 158, which includes strap portion 172 having left end 174 and right end 176. Strap portion 172 is preferably provided on top surface 178 with a portion of a hook and loop fastener that extends from left end 174 to right end 176. Mating portions of a hook and loop fastener are mounted on both left halter support 26 and right halter support 28 at multiple locations, two of which for right halter support 28, rear fastener 180 and front fastener 182, are shown in FIGS. 15B and 15C. Between rear fastener 180 and front fastener 182 is in intermediate fastener (not shown) with which a portion of the hook and loop fastener on strap portion 172 is engaged, after wrapping around right halter support 28, as shown in FIGS. 15B and 15C. In the preferred embodiment of the invention shown in the drawings, fasteners similar to rear fastener 180, front fastener 182 and the intermediate fastener on right halter support 28 are also provided on left halter support 26. Forehead strap 158 also includes forehead pad 184, which is adapted to provide for a comfortable fit of the forehead strap across the patient's forehead as shown in FIG. 15C. The proper placement of suspension strap 156 and forehead strap 158 of head halter 150 on the left and right halter supports is shown in FIGS. 15B and 15C. Left halter support 26, right halter support 28 and head halter 150 are sized and configured so that: (a) when right end strap portion 152 of suspension strap 156 is placed through right halter support slot 58 in right halter support 28 and is secured to the portion of a hook and loop fastener on right halter support 28, and (b) left end strap portion 154 is placed through left halter support slot 58 in left halter support 26 and is secured to the portion of a hook and loop fastener on left halter support 26, and (c) the patient is positioned so that rear edge 166 of suspension strap 156 is at or slightly beneath the occiput, and (d) the forehead strap is place so that forehead pad 184 is on the patient's forehead, and (e) left end 174 of strap portion 172 is securely engaged, after wrapping around right halter support 28, with a hook and loop fastener portion on the right halter support, and (f) right end 176 of strap portion 172 is securely engaged, after wrapping around left halter support 26, with a hook and loop fastener portion on the left halter support, at least a portion of the patient's neck and lower skull will be suspended so that these portions of the patient's anatomy do not contact any other portion or component of the halter assembly or the cervical traction device. In this circumstance, the application of a traction force to the patient by movement of sled assembly 24 on the sled frame will provide a C1-C7 release of the cervical spine.

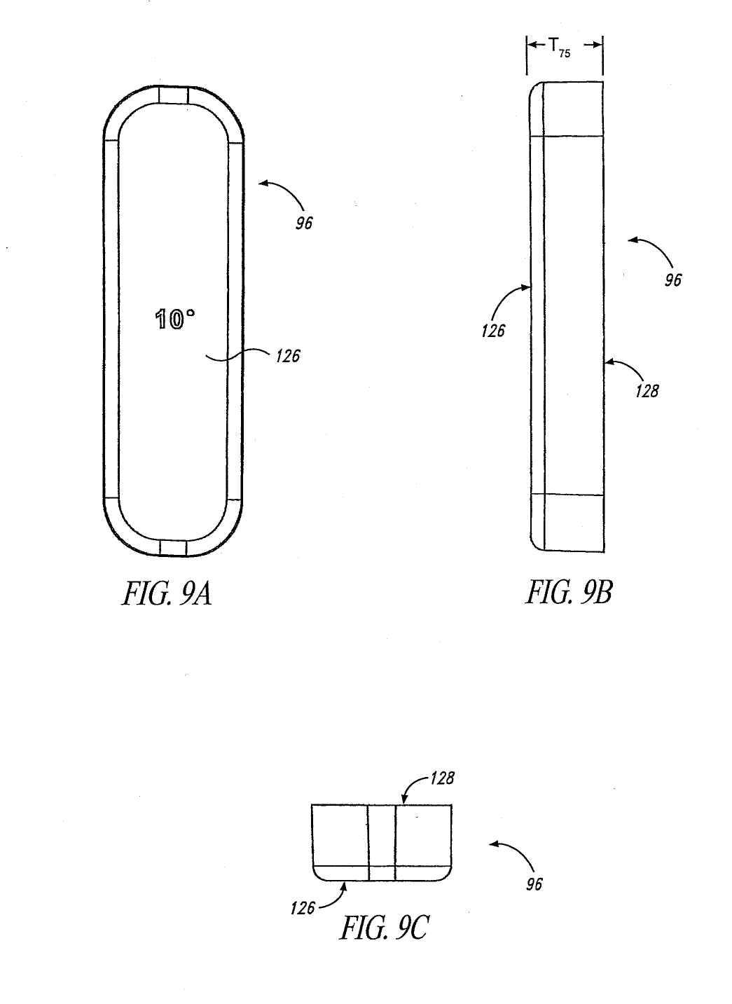

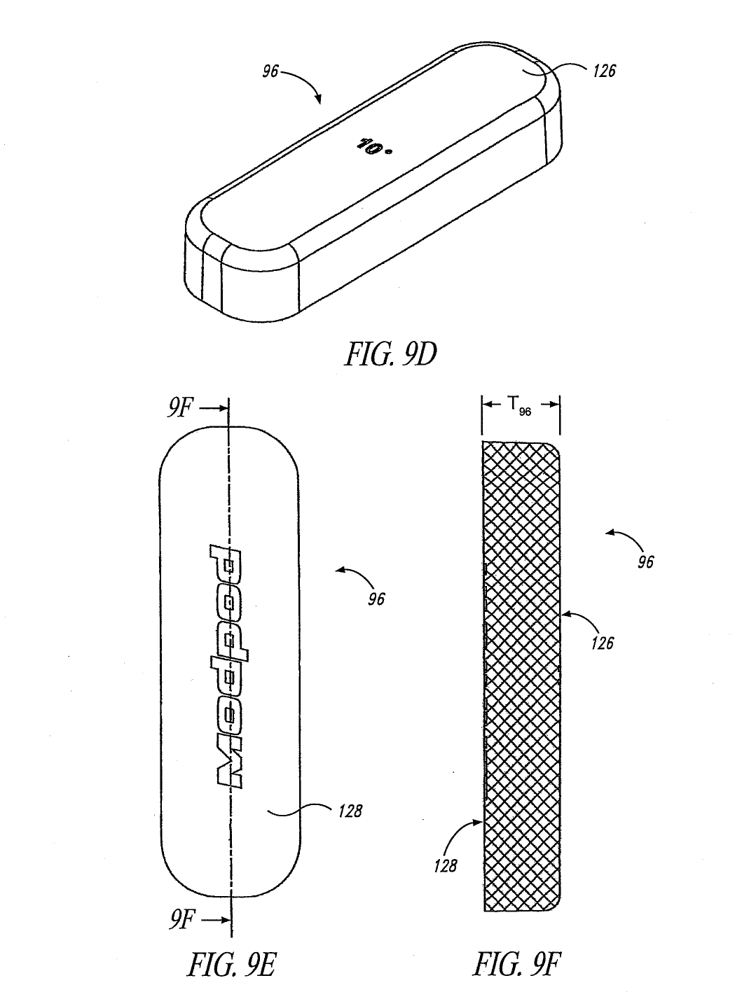

As shown in FIGS. 9A-9F, head support cushion 96 has upper surface 126 that is generally parallel to lower surface 128, so that the thickness T₉₆ is generally constant along the length of the head support cushion. When head support cushion 96 is properly inserted into the recess defined in part by cut-out 94 (as shown in FIG. 1, for example), and a patient is positioned with his shoulders on shoulder support cushion 30, his head on head support cushion 96 between left halter support 26 and right halter support 28, and his neck supported by head halter 62 or head halter 70 as described above, the angle of the traction force applied by movement of sled frame 36 on sled base 54 with respect to the surface on which most of the patient's body rests will be approximately 10°.

As shown in FIGS. 10A-10F, head support cushion 98 has a front end 130 and a rear end 132. The upper surface 134 of head support cushion 98 is inclined upwardly towards front end 130, preferably by 5° with respect to lower surface 136, as best shown in FIG. 10B. Consequently, when head support cushion 98 is properly inserted into the recess defined in party by cut-out 94 with front end 130 towards front end 138 of cut-out 94 (shown in FIG. 8), and a patient is positioned with his shoulders on shoulder support cushion 30, his head on head support cushion 98 between left halter support 26 and right halter support 28, and his neck supported by head halter 62, head halter 70 or head halter 150 as described above, the angle of the traction force applied by movement of sled frame 36 on sled base 54 with respect to the surface on which most of the patient's body rests will be approximately 10°+5°=15°.

As shown in FIGS. 11A-11F, head support cushion 100 has a front end 140 and a rear end 142. The upper surface 144 of head support cushion 100 is inclined upwardly towards front end 140 preferably by 10° with respect to lower surface 146, as best shown in FIG. 11B. Consequently, when head support cushion 100 is properly inserted into the recess defined in part by cut-out 94, with front end 140 towards front end 138 of cut-out 94, and a patient is positioned with his shoulders on shoulder support cushion 30, his head on head support cushion 100 between left halter support 26 and right halter support 28, and his neck supported by head halter 62, head halter 70 or head halter 150 as described above, the angle of the traction force applied by movement of sled frame 36 on sled base 54 with respect to the surface on which most of the patient's body rests will be approximately 10°+10°=20°.

Head support cushions that cooperate with the halter assembly and the shoulder support cushion to provide different angles of application of a traction force from those specifically described herein are contemplated within the scope of the invention.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations as would be appreciated by those having ordinary skill in the art to which the invention relates.

What is claimed is:

1. A cervical traction device for use in applying cervical traction to a patient, said cervical traction device comprising:
(a) a base assembly having a sled frame;
(b) a sled assembly that is adapted to move along the sled frame;
(c) a linear actuator that is:
(i) attached between the base assembly and the sled assembly;
(ii) adapted to move the sled assembly along the sled frame;
(d) a halter assembly comprising:
(i) a left halter support;
(ii) a right halter support;
(iii) a head halter having a suspension strap for supporting the patient's neck and being adapted to be removably attached to the left halter support and the right halter support.

2. The cervical traction device of claim 1 wherein:
(a) the sled assembly includes a sled base;
(b) the left halter support is attached to the sled base;
(c) the right halter support is attached to the sled base.

3. The cervical traction device of claim 1:
(a) which comprises a plurality of head support cushions, each of which has a configuration that is different from the configurations of the other head support cushions;
(b) wherein the sled assembly includes a recess that is adapted to removably receive each of the plurality of head support cushions;
(c) which comprises a shoulder support cushion.

4. The cervical traction device of claim 1 wherein the suspension strap of the head halter is adapted to extend between the left halter support and the right halter support so as to support the patient's neck at or slightly beneath the patient's occiput.

5. The cervical traction device of claim 1 wherein:
(a) the head halter comprises:
(i) a pair of end straps;
(ii) a neck suspension strap having a rear edge;
(iii) a forehead support strap;
(b) each of the halter supports has a slot that is adapted to receive an end strap of the head halter.

6. The cervical traction device of claim 5 wherein the forehead support strap is adjustable and comprises:
(a) a left side portion that is attached to the neck support pad and includes:
(i) a forehead pad;
(ii) a free end that is provided with a portion of a hook and loop fastener that is adapted to engage and mate with a portion of a hook and loop fastener on the left side portion adjacent the forehead pad;
(b) a right side portion that is attached to the neck support strap and terminates in a ring.

7. The cervical traction device of claim 1 wherein:
(a) the head halter comprises:
(i) a suspension strap;
(ii) a chin strap;
(iii) a pair of fastener straps;
(iv) a pair of cupped ends;
(b) each of the halter supports has a front projection that is adapted to receive a cupped end of the head halter.

8. The cervical traction device of claim 1 wherein:
(a) the head halter comprises:
(i) a suspension strap having a right end strap portion that is adapted to be attached to the right halter support and a left end strap portion that is adapted to be attached to the left halter support;
(ii) a forehead strap having a right end that is adapted to be attached to the right halter support and a left end that is adapted to be attached to the left halter support.

9. The cervical traction device of claim 8 wherein:
(a) the right end strap portion of the suspension strap has a portion of a hook and loop fastener;
(b) the left end strap portion of the suspension strap has a portion of a hook and loop fastener;
(c) the right halter support includes:
(i) a right halter support slot that is adapted to receive the right end strap portion of the suspension strap;
(ii) a portion of a hook and loop fastener that is adapted to mate with the portion of the hook and loop fastener on the right end strap portion when the right end strap portion is passed through the right halter support slot;
(d) the left halter support includes:
(i) a left halter support slot that is adapted to receive the left end strap portion of the suspension strap;
(ii) a portion of a hook and loop fastener that is adapted to mate with the portion of the hook and loop fastener on the left end strap portion when the left end strap portion is passed through the left halter support slot;
(e) the forehead strap includes a forehead pad.

10. The cervical traction device of claim 8 wherein a neck pad is attached to the suspension strap.

11. The cervical traction device of claim 10 wherein the neck pad:
(a) has a rear edge;
(b) has a front edge;
(c) comprises an anti-slip fabric material;
(d) includes a semi-rigid flexible material that is sewn into the anti-slip material near the front edge.

12. A method for applying cervical traction to a patient, said method comprising:
(a) providing a cervical traction device comprising:
(i) a base assembly having a sled frame;
(ii) a sled assembly that is adapted to move along the sled frame;
(iii) a linear actuator that is adapted to move the sled assembly along the sled frame;
(iv) a left halter support that is attached to the sled assembly;
(v) a right halter support that is attached to the sled assembly;
(vi) a head halter that is adapted to be removably attached to the left halter support and the right halter support, said head halter having a suspension strap for supporting the patient's neck between the left and right halter supports;
(b) attaching the head halter to the patient's head;
(c) operating the linear actuator to move the sled assembly along the sled frame.

13. A head halter for use with a cervical traction device comprising a base assembly having a sled frame, a sled assembly that is adapted to move along the sled frame, a linear actuator that is adapted to move the sled assembly along the sled frame and a halter assembly including a left halter support that is attached to the sled assembly and a right halter support that is attached to the sled assembly, said head halter including a suspension strap for supporting the patient's neck between the left halter support and the right halter support.

14. The head halter of claim 13:
(a) wherein the suspension strap is adapted to be fastened between the right halter support and the left halter support;
(b) which includes a forehead strap that is adapted to be fastened between the right halter support and the left halter support.

15. The head halter of claim 14:
(a) wherein the suspension strap includes:
(i) a right end strap portion having a portion of a hook and loop fastener;
(ii) a left end strap portion having a portion of a hook and loop fastener;
(b) which is adapted for use with a cervical traction device having:
(i) a right halter support having a right halter support slot that is adapted to receive the right end strap portion of the suspension strap and a portion of a hook and loop fastener that is adapted to mate with the portion of the hook and loop fastener on the right end strap portion when the right end strap portion is passed through the right halter support slot;
(ii) a left halter support having a left halter support slot that is adapted to receive the left end strap portion of the suspension strap and a portion of a hook and loop fastener that is adapted to mate with the portion of the hook and loop fastener on the left end strap portion when the left end strap portion is passed through the left halter support slot;
(c) wherein the forehead strap includes:
(i) a strap portion with a left end and a right end;
(ii) a forehead pad.

16. The head halter of claim 13 which includes a neck pad that is attached to the suspension strap.

17. The head halter of claim 16 wherein the neck pad:
(a) has a rear edge;
(b) has a front edge;
(c) comprises an anti-slip fabric material;
(d) includes a semi-rigid flexible material that is sewn into the anti-slip material near the front edge.

18. The head halter of claim 13:
(a) which is adapted for use with a cervical traction device having a left halter support that includes a left halter support slot;
(b) which includes a left end strap which is adapted to be received in the left halter support slot;
(c) which is adapted for use with a cervical traction device having a right halter support that includes a right halter support slot;
(d) which includes a right end strap which is adapted to be received in the right halter support slot;
(e) which includes a neck suspension strap having a rear edge;
(f) which includes a forehead support strap that is adjustable and comprises:
(i) a left side portion that is attached to the neck suspension pad and includes a forehead pad and a free end that is provided with a portion of a hook and loop fastener that is adapted to engage and mate with a portion of a hook and loop fastener on the left side portion adjacent the forehead pad;
(ii) a right side portion that is attached to the neck suspension strap and terminates in a ring.

19. The head halter of claim 13 which:
(a) includes a back strap;
(b) includes a chin strap;
(c) includes a pair of fastener straps;
(d) includes a pair of cupped ends;
(e) is adapted for use with a cervical traction device having:
(i) a left halter support that is provided with a front projection which is adapted to receive a cupped end of the head halter;
(ii) a right halter support that is provided with a front projection which is adapted to receive a cupped end of the head halter.
